(19) United States
(12) Patent Application Publication
(10) Pub. No.: US 2026/0216499 A1
(43) Pub. Date: Jul. 30, 2026

(54) AI/ML-BASED CLOSED-LOOP SPINAL CORD STIMULATION (SCS)

(71) Applicant: Trishul Kapoor, MOUNTAIN TOP, PA (US)

(72) Inventor: Trishul Kapoor, MOUNTAIN TOP, PA (US)

(21) Appl. No.: 19/038,582

(22) Filed: Jan. 27, 2025

(51) Int. Cl. A61N 1/02; A61N 1/36; A61N 1/372; G16H 40/67; G16H 50/70

(52) U.S. Cl. CPC A61N 1/025; A61N 1/36062; A61N 1/36139; A61N 1/36142; A61N 1/36146; A61N 1/37217; A61N 1/37252; G16H 40/67; G16H 50/70; A61N 1/36071; A61N 1/36114

(57) ABSTRACT

A method comprising receiving, by a machine learning system, evoked compound action potential (ECAP) signals from a spinal cord stimulation device implanted in a patient, wherein the machine learning system continuously monitors biomarkers of the patient through the ECAP signals and automatically modifies stimulation parameters of the spinal cord stimulation device based on the monitored biomarkers, wherein the machine learning system receives Internet of Things (IoT) data from a plurality of IoT devices and trains a machine learning model using the IoT data to generate optimized programming parameters for the spinal cord stimulation device, and wherein the machine learning system clusters patients into groups based on at least one of medical conditions and demographics to generate additional insights for the optimized programming parameters.

### BACKGROUND

Currently, spinal cord stimulation programming is completed in the clinic and, rarely, remotely. Please see the attached article that provides details on SCS programming. SCS has four programmable parameters: contact (electrode) selection (4-16 contacts), amplitude (0-30 mA), pulse width (0-300 μs), and frequency (0-10,000 Hz). Each parameter needs to be accounted for when assessing which program works for which patient. Traditional open-loop systems allow for different “programs,” or combinations of these four parameters, to be pre-set by the provider and medical device representative. These allow for flexibility in the type of stimulation delivered to the patient depending on activity. Patients are also given control over programs and changing the amplitudes of these programs. However, some open-loop systems place the burden of toggling between programs to manage pain control on patients. Newer closed-loop systems make it possible for stimulation settings to automatically adjust in response to accelerometry and evoked compound action potential feedback and therefore have the potential to streamline the patient

experience.

### BRIEF SUMMARY OF THE INVENTION

In one aspect, a system comprising an implantable pulse generator configured to deliver spinal cord stimulation therapy to a patient, wherein a plurality of leads are coupled to the implantable pulse generator, and wherein a machine learning module is configured to receive evoked compound action potential (ECAP) signals from the implantable pulse generator, continuously monitor biomarkers of the patient through the ECAP signals, automatically modify stimulation parameters of the implantable pulse generator based on the monitored biomarkers, receive Internet of Things (IoT) data from a plurality of IoT devices, train a machine learning model using the IoT data to generate optimized programming parameters for the implantable pulse generator, and cluster patients into groups based on at least one of medical conditions and demographics to generate additional insights for the optimized programming parameters.

In another aspect, a method comprising receiving, by a machine learning system, evoked compound action potential (ECAP) signals from a spinal cord stimulation device implanted in a patient, wherein the machine learning system continuously monitors biomarkers of the patient through the ECAP signals and automatically modifies stimulation parameters of the spinal cord stimulation device based on the monitored biomarkers, wherein the machine learning system receives Internet of Things (IoT) data from a plurality of IoT devices and trains a machine learning model using the IoT data to generate optimized programming parameters for the spinal cord stimulation device, and wherein the machine learning system clusters patients into groups based on at least one of medical conditions and demographics to generate additional insights for the optimized programming parameters.

### BRIEF DESCRIPTION OF THE DRAWINGS

FIG. 1 illustrates an example process for AI/ML-based closed-loop spinal cord stimulation (SCS), according to some embodiments.

FIG. 2 illustrates an example ML system for closed-loop SCS optimization, according to some embodiments.

FIG. 3 illustrates an example process for using IoT data integration to enhance a closed-loop SCS ML model, according to some embodiments.

FIG. 4 illustrates an example process for implementing a ML Model Integration Framework, according to some embodiments.

FIG. 5 illustrates an example implantable pulse generator with an AI/ML-based SCS module, according to some embodiments.

The Figures described above are a representative set and are not an exhaustive with respect to embodying the invention.

### DESCRIPTION

Disclosed are a system, method, and article of manufacture for AI/ML-based closed-loop spinal cord stimulation (SCS). The following description is presented to enable a person of ordinary skill in the art to make and use the various embodiments. Descriptions of specific devices, techniques, and applications are provided only as examples. Various modifications to the examples described herein can be readily apparent to those of ordinary skill in the art, and the general principles defined herein may be applied to other examples and applications without departing from the spirit and scope of the various embodiments.

Reference throughout this specification to ‘one embodiment,’ ‘an embodiment,’ ‘one example,’ or similar language means that a particular feature, structure, or characteristic described in connection with the embodiment is included in at least one embodiment, according to some embodiments. Thus, appearances of the phrases ‘in one embodiment,’ ‘in an embodiment,’ and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art can recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, and they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

### Definitions

Artificial cardiac pacemaker, commonly referred to as simply a pacemaker, is an implanted medical device that generates electrical pulses delivered by electrodes to one or more of the chambers of the heart. Each pulse causes the targeted chamber(s) to contract and pump blood, thus regulating the function of the electrical conduction system of the heart.

Cluster analysis or clustering is the task of grouping a set of objects in such a way that objects in the same group (e.g. a cluster) are more similar (e.g. in some specific sense defined by the analyst) to each other than to those in other groups (e.g. clusters).

Evoked Compound Action Potential (ECAP) is an electrical signal that represents the collective response of multiple nerve fibers firing simultaneously in response to an electrical stimulus. In the context of spinal cord stimulation, ECAPs are recorded signals that show the combined activity of many neurons responding to the electrical pulses delivered by the stimulator. In some examples, when an electrical pulse is delivered to the spinal cord through the stimulation electrodes, it activates nearby nerve fibers. These activated nerve fibers then generate their own electrical signals as they conduct the impulse. The ECAP is the sum of all these individual nerve fiber responses, creating a characteristic waveform that can be measured by recording electrodes. ECAPs can provide direct feedback about neural activation. The amplitude of the ECAP correlates with how many nerve fibers were activated. The timing and shape of the ECAP can indicate which types of nerve fibers responded. They can be measured in real-time, making them valuable for closed-loop stimulation systems. ECAPs can provide immediate, objective feedback about how effectively the stimulation is activating the target neural populations, allowing for automated adjustments to optimize therapy delivery.

Implanted pulse generator (IPG) (e.g. neurostimulator) can be a battery-powered device designed to deliver electrical stimulation to the brain and/or other aspect of the nervous system. In some examples, an IPG is a battery-powered micro-electronic device that is implanted in the body and provide electrical stimulation to the nervous system. An IPG can be used for various medical functions (e.g. regulating heart rhythms, managing chronic pain, etc.). An IPG can include electric circuits and a battery. Leads (e.g. wires) are implanted and provide the electrical stimulation to a specified portion of the nervous system. For example, leads inside veins to carry electrical pulses to the heart, helping it to beat in a specific pattern/frequency.

Internet of things (IoT) describes devices with sensors, processing ability, software and other technologies that connect and exchange data with other devices and systems over the Internet or other communication networks.

Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity, and metric learning, and/or sparse dictionary learning.

Spinal Cord Stimulation (SCS) is the application of electrical impulses to the spinal cord through implanted wires and an implantable pulse generator to interrupt pain signals to the brain. There are numerous conditions that are currently approved for SCS treatment (i.e. failed back surgery syndrome, complex regional pain syndrome, post-herpetic neuralgia, chronic painful peripheral neuropathy, multiple sclerosis, angina, arachnoiditis, phantom limb pain, intercostal neuralgia, cauda equina injury, incomplete spinal cord injury, etc.). additionally discussions of examples SCS systems and methods are provided infra.

Example definitions for some embodiments are now provided. These example definitions can be integrated into respective example embodiments discussed infra. These example definitions can be integrated into example embodiments of the systems and methods discussed herein.

### Example Systems and Methods

In some embodiments, Spinal Cord Stimulation (SCS) can be a medical treatment that involves implanting a small device called a neurostimulator near the spine to deliver mild electrical impulses to specific nerves along the spinal cord. These electrical signals work by interrupting or masking pain signals traveling between the spinal cord and the brain, effectively reducing the perception of chronic pain in targeted areas of the body. The implantation can include a minimally invasive surgery to place the neurostimulator, usually in the lower back or buttocks area, along with thin wires (e.g. leads) that carry the electrical impulses to the spinal cord. Patients can control the strength and location of stimulation using an external remote control, allowing them to adjust the therapy based on their pain levels and activities throughout the day.

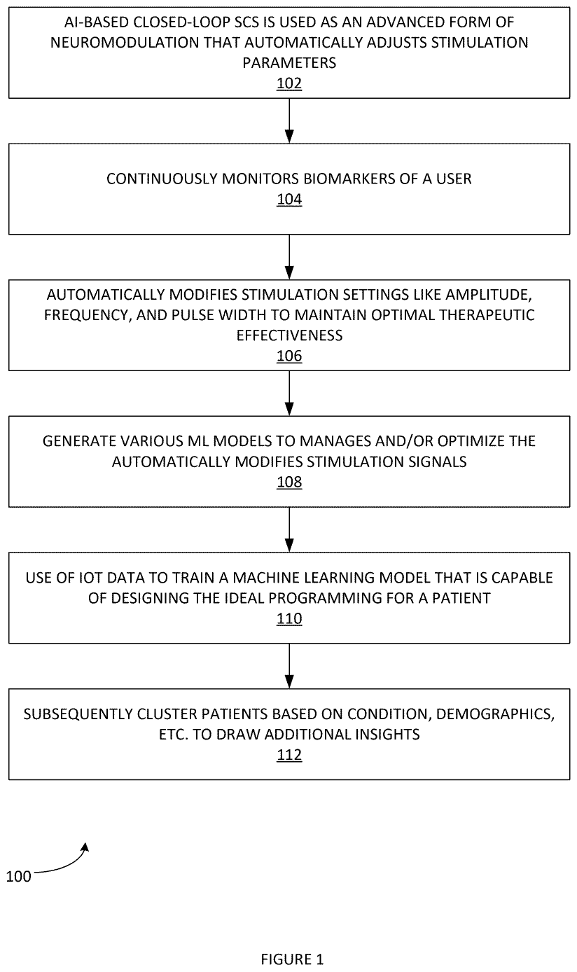

FIG. 1 illustrates an example process 100 for AI/ML-based closed-loop spinal cord stimulation (SCS), according to some embodiments. In step 102, AI-based closed-loop SCS is used as an advanced form of neuromodulation that automatically adjusts stimulation parameters in real-time based on direct feedback from the patient's physiological responses. In some embodiments, a AI-based closed-loop is an advanced form of neuromodulation that automatically adjusts stimulation parameters in real-time based on direct feedback from the patient's physiological responses.

In step 104, process 100 continuously monitors biomarkers of a user. These can include ECAPs and/or local field potentials from the spinal cord, which serve as objective measures of neural activation. When these measurements indicate changes in the patient's pain state or position, process 100 automatically modifies stimulation settings like amplitude, frequency, and pulse width to maintain optimal therapeutic effectiveness in step 106. Closed-loop SCS creates a dynamic therapeutic environment that adapts to the patient's changing needs throughout the day.

In step 108, process 100 can generate various ML models to manages and/or optimize the automatically modifies stimulation signals. This can be done automatically, dynamically and/or in real time (e.g. assuming networking and computational latencies, etc.). In this way, process 100 applies artificial intelligence or machine learning in SCS programming. In step 110, process 100 can use of IoT data (e.g. video, audio, environmental, health, location, smart home, traffic data, etc.) to train a machine learning model that is capable of designing the ideal programming for a patient.

In step 112, process 100 can then subsequently cluster patients based on condition, demographics, etc. to draw additional insights. Process 100 can generally leads to more consistent pain relief while using less energy, which can extend battery life and reduce the need for manual reprogramming sessions.

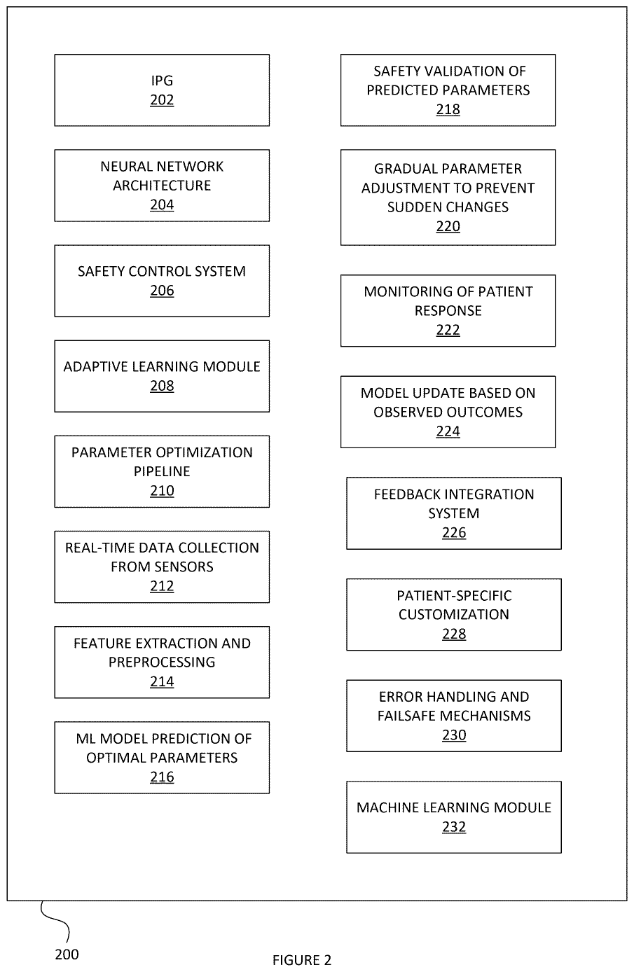

FIG. 2 illustrates an example ML system 200 for closed-loop SCS optimization, according to some embodiments. ML system 200 can be structured in several interconnected modules. ML system 200 includes an Input Processing Module 202. Input Processing Module 202 can continuously monitors three primary data streams. These can include, inter alia: ECAP (Evoked Compound Action Potentials) signals from the spinal cord, positional data from an embedded accelerometer, and current stimulation parameters. These inputs can be preprocessed using signal processing techniques to remove noise and extract relevant features. The ECAP signals can be processed in small time windows to capture temporal patterns in neural responses.

Neural Network Architecture 204 can use a hybrid neural network combining LSTM (Long Short-Term Memory) layers for processing temporal ECAP patterns with dense layers for handling static inputs. This example architecture allows the ML system 200 to learn both immediate responses and longer-term patterns in patient responses to stimulation. The network outputs optimized values for amplitude, frequency, pulse width, and electrode configuration. It is noted that other ML methodologies can be utilized in other example embodiments (e.g. see those discussed infra, etc.).

Safety Control System 206 can manage a safety monitoring system that acts as a supervisory layer. Safety Control System 206 can implement various hard constraints on stimulation parameters, preventing the ML model from generating potentially harmful stimulation patterns. This can include maximum limits on, inter alia: amplitude, frequency, and pulse width, as well as rules for safe electrode configuration transitions. Safety Control System 206 can maintain a log of all parameter adjustments and any safety interventions.

IPG Neural Network Architecture Safety Control System Adaptive Learning Module 208 can implement online learning capabilities, allowing it to continuously refine its predictions based on patient responses. It maintains a rolling window of recent stimulation outcomes and adjusts its parameters using reinforcement learning techniques. It is noted that Adaptive Learning Module 208 can implemented on a local implanted device as well in whole or in part. The learning rate is dynamically adjusted based on the confidence in current predictions and the stability of patient responses.

Parameter Optimization Pipeline 210 provides an optimization process follows a structured pipeline. For example, Real-time data collection from sensors 212, Feature extraction and preprocessing 214, ML model prediction of optimal parameters 216, Safety validation of predicted parameters 218, Gradual parameter adjustment to prevent sudden changes 220, Monitoring of patient response 222, Model update based on observed outcomes, 224. Examples of these are now provided.

Real-time data collection from sensors 212 is now discussed. ML system 200 can continuously gather streaming data from multiple sources including ECAP signals from the stimulator electrodes, positional information from accelerometers, and physiological data from integrated sensors. This data collection occurs at high sampling rates (typically hundreds to thousands of times per second) to capture rapid neural responses and physical changes with minimal latency.

Feature extraction and preprocessing 214 is now discussed. Raw sensor data is processed to extract meaningful features while removing noise, artifacts, and irrelevant information through techniques like filtering, normalization, and signal decomposition. The extracted features might include ECAP waveform characteristics, movement patterns, and physiological markers that are then formatted into standardized inputs for the ML model.

ML model prediction of optimal parameters 216 is now discussed. The trained machine learning model uses the preprocessed features to predict the optimal stimulation parameters (e.g. amplitude, frequency, pulse width, and electrode configuration) for the current patient state. The predictions are made using a hybrid neural network that combines temporal pattern analysis with current contextual information to generate parameter recommendations in millisecond timeframes.

Safety validation of predicted parameters 218 is now discussed. Each set of predicted stimulation parameters undergoes rigorous safety checks against predetermined therapeutic bounds and physiological limits to ensure they fall within safe operating ranges. The validation system also checks for potentially harmful parameter combinations and ensures that the proposed changes comply with medical safety guidelines and patient-specific restrictions.

Gradual parameter adjustment to prevent sudden changes 220 is now discussed. Rather than immediately implementing predicted parameter changes, ML system 200 can employ a ramping mechanism that smoothly transitions from current to target parameters over an appropriate timeframe. This gradual adjustment helps prevent patient discomfort and allows the system to monitor for adverse responses during the transition.

Monitoring of patient response 222 is now discussed. ML system can continuously track multiple indicators of therapy effectiveness, including direct neural responses through ECAP measurements and indirect measures like movement patterns or physiological markers. The monitoring system maintains ongoing vigilance for both positive therapeutic outcomes and potential adverse effects that might require parameter adjustments.

Model update based on observed outcomes 224 is now discussed. The machine learning model undergoes periodic retraining using accumulated patient response data to improve its prediction accuracy and adapt to changes in patient condition or therapy requirements. The model updates incorporate both successful and unsuccessful parameter adjustments to refine the system's understanding of optimal stimulation patterns for the specific patient.

Feedback Integration System 226 can incorporate both direct feedback (e.g. ECAP signals) and indirect feedback (e.g. position changes, therapy outcomes, etc.) to create a comprehensive understanding of treatment efficacy. This multi-modal feedback approach helps in creating a more robust and personalized therapy delivery.

Patient-Specific Customization 228 can maintains individual patient profiles that include historical response patterns, preferred stimulation ranges, and specific contraindications. These profiles influence the parameter optimization process and help in maintaining personalized therapy delivery.

Error Handling and Failsafe Mechanisms 230 provides robust error detection and handling mechanisms. If anomalies are detected in either the input signals or the model's predictions, it can fall back to safe, pre-programmed stimulation patterns while alerting the medical team.

This ML system is designed to operate in real-time within the constraints of implanted hardware, with emphasis on safety, reliability, and effectiveness. The system periodically saves its state and can be remotely monitored by healthcare providers for performance assessment and manual adjustment if needed.

Machine Learning Module 232 can manage and implement the various machine learning operations discussed herein. Machine Learning Module 232 can implement one or more alternatives to Neural Network Architecture 204. Machine Learning Module 232 can implement other ML functionalities and methods other than those already provided. Machine learning is a type of artificial intelligence (AI) that provides computers with the ability to learn without being explicitly programmed. Machine learning focuses on the development of computer programs that can teach themselves to grow and change when exposed to new data. Example machine learning techniques that can be used herein include, inter alia: decision tree learning, association rule learning, artificial neural networks, inductive logic programming, support vector machines, clustering, Bayesian networks, reinforcement learning, representation learning, similarity, and metric learning, and/or sparse dictionary learning. Random forests (RF) (e.g., random decision forests) are an ensemble learning method for classification, regression, and other tasks, which operate by constructing a multitude of decision trees at training time and outputting the class that is the mode of the classes (e.g., classification) or mean prediction (e.g., regression) of the individual trees. RFs can correct for decision trees'habit of overfitting to their training set. Deep learning is a family of machine learning methods based on learning data representations. Learning can be supervised, semi-supervised or unsupervised.

Machine learning can be used to study and construct algorithms that can learn from and make predictions on data. These algorithms can work by making data-driven predictions or decisions, through building a mathematical model from input data. The data used to build the final model usually comes from multiple datasets. In particular, three data sets are commonly used in different stages of the creation of the model. The model is initially fit on a training dataset, which is a set of examples used to fit the parameters (e.g., weights of connections between neurons in artificial neural networks) of the model. The model (e.g., a neural net or a naive Bayes classifier) is trained on the training dataset using a supervised learning method (e.g., gradient descent or stochastic gradient descent). In practice, the training dataset often consist of pairs of an input vector (or scalar) and the corresponding output vector (or scalar), which is commonly denoted as the target (or label). The current model is run with the training dataset and produces a result, which is then compared with the target, for each input vector in the training dataset. Based on the result of the comparison and the specific learning algorithm being used, the parameters of the model are adjusted. The model fitting can include both variable selection and parameter estimation. Successively, the fitted model is used to predict the responses for the observations in a second dataset called the validation dataset. The validation dataset provides an unbiased evaluation of a model fit on the training dataset while tuning the model's hyperparameters (e.g., the number of hidden units in a neural network). Validation datasets can be used for regularization by early stopping: stop training when the error on the validation dataset increases, as this is a sign of overfitting to the training dataset. Finally, the test dataset is a dataset used to provide an unbiased evaluation of a final model fit on the training dataset. If the data in the test dataset has never been used in training (e.g., in cross-validation), the test dataset is also called a holdout dataset.

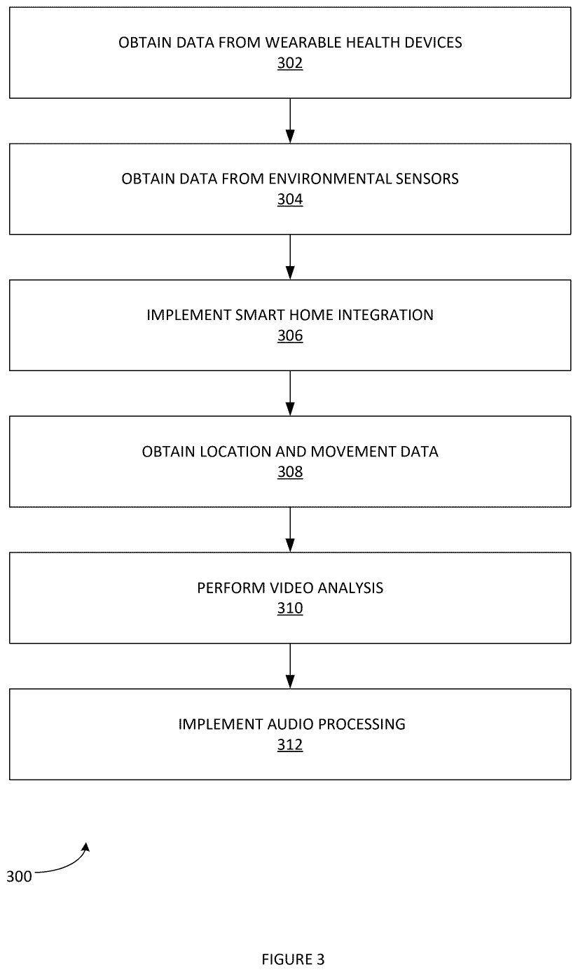

FIG. 3 illustrates an example process 300 for using IoT data integration to enhance a closed-loop SCS ML model, according to some embodiments. Process 300 can be used to enhance process 100 and/or ML system 200 by way of example. For example, an IoT data integration could enhance the closed-loop SCS machine learning model. Process 300 can obtain IoT data from contextual IoT Data Sources. In step 302, process 300 can obtain data from Wearable Health Devices. Process 300 can obtain heart rate variability and ECG patterns to correlate cardiovascular stress with pain levels. Process 300 can obtain sleep quality data to understand pain patterns during rest. Process 300 can obtain blood pressure readings to detect stress responses. Process 300 can obtain activity levels from fitness trackers to gauge mobility. Process 300 can obtain skin conductance for stress response measurement.

Process 300 can obtain data from Environmental Sensors in step 304. These can include barometric pressure changes that might affect pain levels. These can include temperature and humidity data that could influence inflammation. These can include air quality metrics that might impact overall well-being. These can include light levels to track circadian rhythm impact on pain.

In step 306, process 300 can implement Smart Home Integration. Process 300 can obtain data from bed sensors for sleep position and movement patterns. Process 300 can obtain data from smart chair sensors for posture analysis. Process 300 can obtain data from room occupancy patterns to track daily activity levels. Process 300 can obtain data from smart thermostat data to correlate temperature preferences with pain.

In step 308, process 300 can obtain 310 Location and Movement data. Process 300 can obtain data GPS data to understand pain patterns in different locations. Process 300 can obtain data from accelerometer data from phone for broader movement analysis. Process 300 can obtain data from transportation mode detection sources (e.g. walking, driving, public transit). Process 300 can obtain data from indoor positioning data sources to track home/office movement patterns.

In step 310, process 300 can perform Video Analysis. This can include gait analysis from home security cameras. This can include posture assessment from dedicated monitoring cameras. This can include facial expression analysis for pain level estimation. This can also include movement pattern analysis in daily activities.

In step 312, process 300 can implement Audio Processing. Voice stress analysis can be performed for pain assessment; vocal biomarkers of discomfort; ambient noise levels that might affect stress; speech pattern changes indicating pain levels; etc. Process 300 can provide IoT data and relevant contextual analysis to process 400.

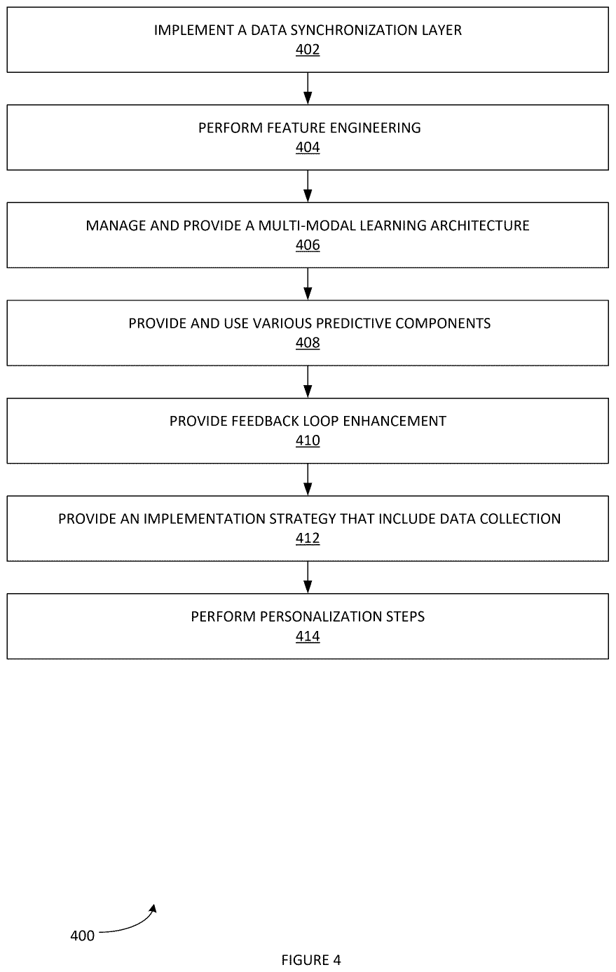

FIG. 4 illustrates an example process 400 for implementing a ML Model Integration Framework, according to some embodiments. Process 400 can process the output of process 300. In step 402, process 400 implements a Data Synchronization Layer. Here, process 400 performs time-stamping all IoT data streams. Data Synchronization Layer provides alignment with SCS signal data. Data Synchronization Layer provides data quality validation. Data Synchronization Layer performs missing data handling.

In step 404, process 400 performs Feature Engineering. Feature Engineering includes extraction of temporal patterns. Feature Engineering includes cross-device correlation analysis. Feature Engineering include context-aware feature generation. Feature Engineering includes dimensionality reduction for efficient processing.

In step 406, process 400 manages and provides a multi-modal learning architecture. Multi-modal learning architecture can implement parallel processing streams for different data types. Multi-modal learning architecture provides attention mechanisms for important feature selection. Multi-modal learning architecture implements temporal pattern recognition across data streams. Multi-modal learning architecture implement context-aware parameter optimization.

In step 408, process 400 provides and uses various Predictive Components. Predictive Components can include, inter alia: pain level prediction from environmental factors; activity-specific stimulation parameter adjustment; preemptive parameter modification based on context; personalized rhythm detection and adaptation; etc.

In step 410, process 400 provides Feedback Loop Enhancement. This can include, inter alia: real-time correlation of IoT data with pain relief; pattern detection for optimal parameter settings; environmental trigger identification; Activity-based effectiveness measurement; etc.

In step 412, process 400 provides an Implementation Strategy that include Data Collection. Data collection can be performed via, inter alia: secure data collection from IoT devices; privacy-preserving data processing; edge computing for sensitive data; bandwidth-efficient data transmission; etc. This step can include a processing pipeline for real-time data streaming architecture and/or edge processing for immediate response. Cloud processing for long-term pattern analysis can be performed as well.

In step 414, process 400 performs Model Optimization. Context-specific parameter adjustment can be performed. Activity-based stimulation profiles can be performed. Environmental condition adaptation can be performed. Personalized pattern recognition can be implemented.

Process 400 can perform various other option functions. These can include Safety and Validation by cross-validation with multiple data sources and/or anomaly detection across IoT streams. Safety boundary enforcement can be present. Data quality assurance steps can be utilized as well.

In step 414, process can perform personalization steps. These can include, inter alia: individual activity pattern learning; environment-specific response profiles; daily routine adaptation; lifestyle-based optimization; etc.

Process 400 can create a more comprehensive understanding of pain patterns and treatment efficacy by incorporating real-world contextual data, allowing for more precise and personalized stimulation parameter optimization.

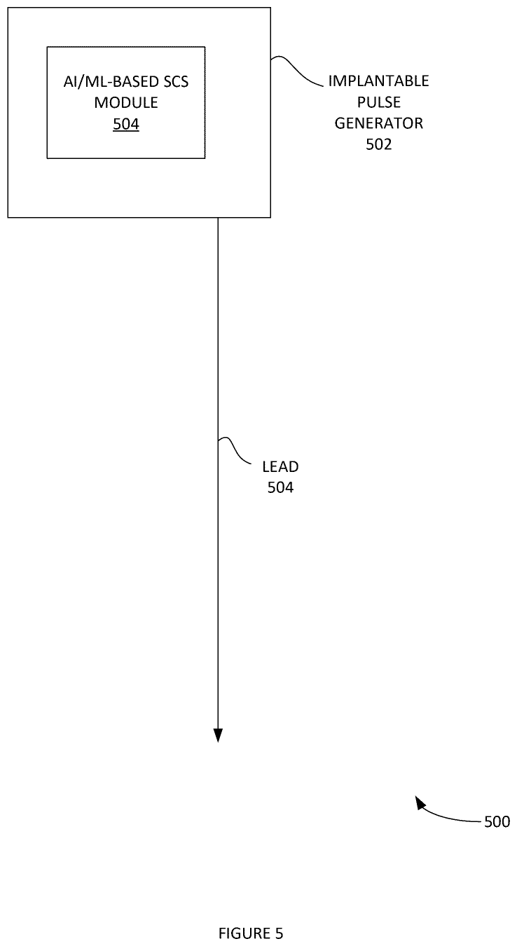

FIG. 5 illustrates an example implantable pulse generator with an AI/ML-based SCS module, according to some embodiments.

In some examples, an IPG 502 can be a battery-powered micro-electronic device that is implanted in the body and provide electrical stimulation to the nervous system. IPG 502 can be used for various medical functions (e.g. regulating heart rhythms, managing chronic pain, etc.). IPG 302 can include electric circuits and a battery. Optionally, IPG 202 can include process 100, ML system 200, processes 300-400 in whole or in part according to various embodiments via AI/ML-based SCS module 504. AI/ML-based SCS module 504 can also connect wirelessly to a computing system that offloads aspects of these functions. AI/ML-based SCS module 504 thus implements and manages AI/ML SCS in IPG 502. AI/ML-based SCS module 504 include ML system and obtain IoT data from remote computing devices such as those described supra as a user moves to come within range of these IoT systems.

Lead(s) 506 (e.g. wires) are implanted and provide the electrical stimulation to a specified portion of the nervous system. For example, lead(s) 506 inside veins to carry electrical pulses to the heart, helping it to beat in a specific pattern/frequency. In one example, IPG 502 can include a wireless communication system (e.g. Wi-Fi, Bluetooth®, etc.). In one example, Lead(s) 208 and/or IPG 202 can include various sensors and/or biosensors to obtain data about the user and/or the user's environment.

### Conclusion

Although the present embodiments have been described with reference to specific example embodiments, various modifications and changes can be made to these embodiments without departing from the broader spirit and scope of the various embodiments. For example, the various devices, modules, etc. described herein can be enabled and operated using hardware circuitry, firmware, software or any combination of hardware, firmware, and software (e.g. embodied in a machine-readable medium).

In addition, it can be appreciated that the various operations, processes, and methods disclosed herein can be embodied in a machine-readable medium and/or a machine accessible medium compatible with a data processing system (e.g., a computer system), and can be performed in any order (e.g., including using means for achieving the various operations). Accordingly, the specification and drawings are to be regarded in an illustrative rather than a restrictive sense. In some embodiments, the machine-readable medium can be a non-transitory form of machine-readable medium.

What is claimed by united states patent:

1. A method comprising:
receiving, by a machine learning system, evoked compound action potential (ECAP) signals from a spinal cord stimulation device implanted in a patient;
continuously monitoring, by the machine learning system, biomarkers of the patient through the ECAP signals;
automatically modifying, by the machine learning system, stimulation parameters of the spinal cord stimulation device based on the monitored biomarkers;
receiving Internet of Things (IoT) data from a plurality of IoT devices;
training a machine learning model using the IoT data to generate optimized programming parameters for the spinal cord stimulation device; and
clustering patients into groups based on at least one of medical conditions and demographics to generate additional insights for the optimized programming parameters.

2. The method of claim 1, wherein the stimulation parameters comprise at least one of amplitude, frequency, pulse width, and electrode configuration.

3. The method of claim 1, wherein the IoT devices comprise at least one of wearable health devices, environmental sensors, smart home devices, location sensors, video devices, and audio devices.

4. The method of claim 1, further comprising:
implementing a safety control system that enforces constraints on the stimulation parameters to prevent harmful stimulation patterns.

5. The method of claim 1, further comprising:
implementing an adaptive learning module that continuously refines predictions based on patient responses using reinforcement learning techniques.

6. The method of claim 1, wherein the IoT data comprises at least one of heart rate variability, sleep quality data, blood pressure readings, activity levels, skin conductance, environmental temperature, humidity data, air quality metrics, and light levels.

7. The method of claim 1, further comprising:
performing feature engineering on the IoT data to generate context-aware features for the machine learning model.

8. A system comprising:
an implantable pulse generator configured to deliver spinal cord stimulation therapy to a patient;
a plurality of leads coupled to the implantable pulse generator; a machine learning module configured to:
receive evoked compound action potential (ECAP) signals from the implantable pulse generator; continuously monitor biomarkers of the patient through the ECAP signals;
automatically modify stimulation parameters of the implantable pulse generator based on the monitored biomarkers;
receive Internet of Things (IoT) data from a plurality of IoT devices;
train a machine learning model using the IoT data to generate optimized programming parameters for the implantable pulse generator; and
cluster patients into groups based on at least one of medical conditions and demographics to generate additional insights for the optimized programming parameters.

9. The system of claim 8, wherein the stimulation parameters comprise at least one of amplitude, frequency, pulse width, and electrode configuration.

10. The system of claim 8, wherein the IoT devices comprise at least one of wearable health devices, environmental sensors, smart home devices, location sensors, video devices, and audio devices.

11. The system of claim 8, wherein the machine learning module further comprises: a safety control system that enforces constraints on the stimulation parameters to prevent harmful stimulation patterns.

12. The system of claim 8, wherein the machine learning module further comprises:
an adaptive learning module that continuously refines predictions based on patient responses using reinforcement learning techniques.

13. The system of claim 8, wherein the IoT data comprises at least one of heart rate variability, sleep quality data, blood pressure readings, activity levels, skin conductance, environmental temperature, humidity data, air quality metrics, and light levels.

14. The system of claim 8, wherein the machine learning module is further configured to:
perform feature engineering on the IoT data to generate context-aware features for the machine learning model.

15. The system of claim 8, wherein the implantable pulse generator includes an embedded AI/ML-based spinal cord stimulation module that implements at least a portion of the machine learning module locally within the implantable pulse generator.
